(19) United States
(12) Patent Application Publication
(10) Pub. No.: US 2007/0004025 A1
(43) Pub. Date: Jan. 4, 2007

(54) Replaceable annular electrode for transgenic

(76) Inventors: Junne-Jih Chen, Hsinshe Township (TW); Fang-Sheng Wu, Hsinshe Township (TW); Yung-Wei Sun, Hsinshe Township (TW); Han-Tsu Shen, Hsinshe Township (TW)

Correspondence Address: Connolly Bove Lodge & Hutz LLP, P.O. Box 2207, Wilmington, DE, 19899-2207, US

(21) Appl. No.: 11/170,986

(22) Filed: Jun. 30, 2005

(51) Int. Cl. C12M 1/42

(52) U.S. Cl. 435/283.1; 435/285.2

(57) ABSTRACT

A replaceable annular electrode for transgenic includes a hand-held electrode seat having multiple metal rods downward extending from the hand-held seat. An electrode ring is secured on a free end of each of the multiple metal rods. A first electrode rod centrally downward extends from the hand-held seat and a sleeve is sleeved on the first electrode rod. The sleeve is secured on the first electrode rod by bolts. The sleeve has a second electrode rod centrally downward extending therefrom and the second electrode rod is electrically connected to the first electrode rod when the sleeve is secured on the first electrode rod such that the second electrode rod can be replaced to have various lengths and diameters such that annular electrode can use different distances between the second electrode rod and the electrode ring to make different obvious electric shock effects.

### BACKGROUND OF THE INVENTION

1. Field of the Invention

The present invention relates to an annular electrode, and more particularly to a replaceable annular electrode that is provided for transgenic.

2. Description of Related Art

The known plant transgenic method includes tubularlarrium-mediated transformation, gene gun, electroporation, microinjection, heat shock, pollen tube pathway, etc. The electroporation exposes cells under a condition of a twinkle high-voltage discharge. As a result, multiple tiny bores are temporarily defined in the cell membrane and the plasmid deoxyribonucleic acid (DNA) can get into the cell for achieving the purpose of transgenic. However, the conventional electroporation apparatus usually uses disposable tubular electrode and the plant tissue solution is received in the tubular electrode. In addition, a lower end of the tubular electrode is clamped by an electrode clamp.

However, the conventional electroporation apparatus is unsuitable to a great plant tissue and the room in the tubular electrode is cramped such that the plant tissue may be hurt in the cramped tubular electrode. In addition, the operation of the conventional electroporation apparatus takes a lot of time, costs a lot of money and the tubular electrode can not be repeatedly used. Furthermore, the aluminum foil conducting end is easily oxidized and causes poison material.

The present invention has arisen to mitigate and/or obviate the disadvantages of the conventional tubular electrode.

### SUMMARY OF THE INVENTION

The main objective of the present invention is to provide an improved annular electrode for transgenic that has a replaceable electrode rod.

To achieve the objective, the annular electrode in accordance with the present invention comprises a hand-held electrode seat having multiple metal rods downward extending from the hand-held seat. An electrode ring is secured on a free end of each of the multiple metal rods. A first electrode rod centrally downward extends from the hand-held seat and a sleeve is sleeved on the first electrode rod. The sleeve is secured on the first electrode rod by bolts. The sleeve has a second electrode rod centrally downward extending therefrom and the second electrode rod is electrically connected to the first electrode rod when the sleeve is secured on the first electrode rod such that the second electrode rod can be replaced to have various lengths and diameters such that annular electrode can use different distances between the second electrode rod and the electrode ring to make different obvious electric shock effects.

Further benefits and advantages of the present invention will become apparent after a careful reading of the detailed description with appropriate reference to the accompanying drawings.

### BRIEF DESCRIPTION OF THE DRAWINGS

FIG. 1 is a perspective schematic view of a replaceable annular electrode for transgenic in accordance with the present invention;

FIG. 2 is an exploded perspective view of the replaceable annular electrode in FIG. 1;

FIG. 3 is a partially cross-sectional view of the replaceable annular electrode in FIG. 1; and

FIG. 4 is an operational view in cross-section of the replaceable annular electrode of the present invention.

### DETAILED DESCRIPTION OF THE INVENTION

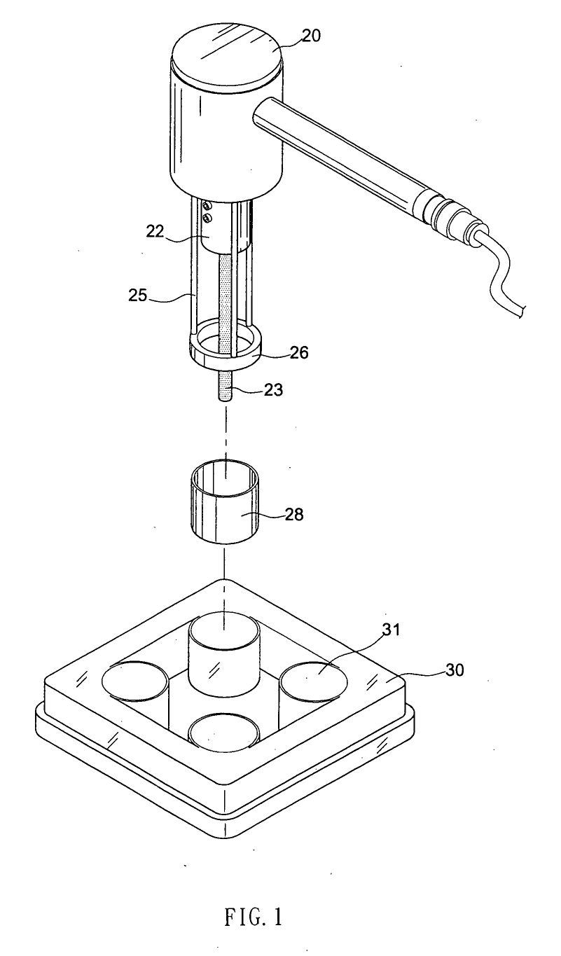

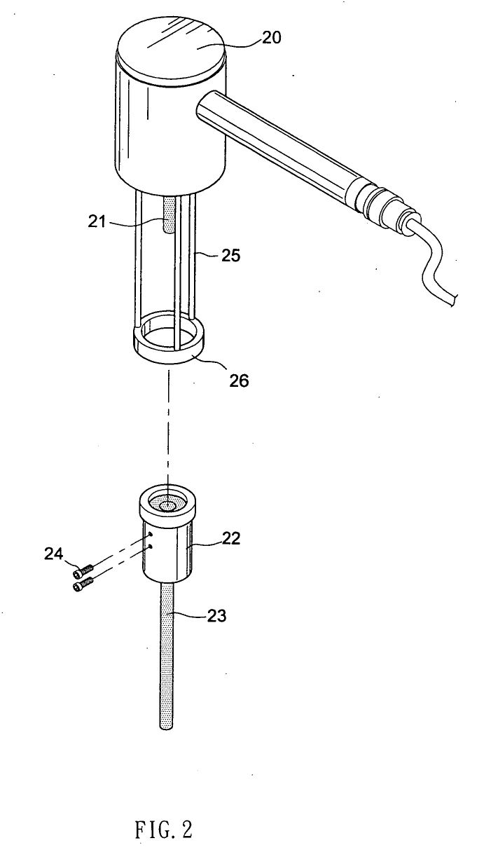

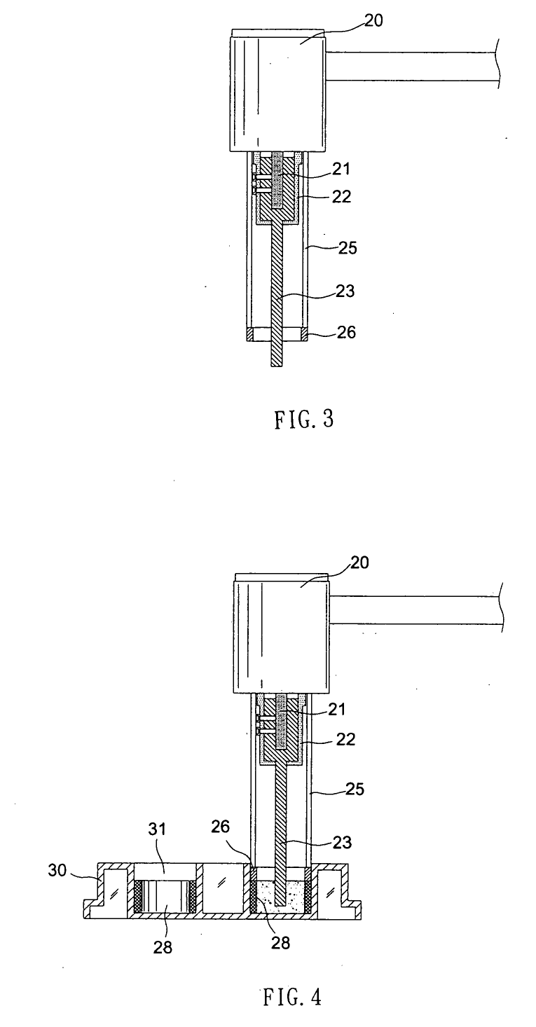

Referring to the drawings and initially to FIGS. 1-4, a replaceable annular electrode for transgenic in accordance with the present invention comprises a hand-held electrode seat (20) having multiple metal rods (25) downward extending therefrom. An electrode ring (26) is secured on a free end of each of the multiple metal rods (25). A first electrode rod (21) centrally downward extends from the hand-held seat (20) and a sleeve (22) is sleeved on the first electrode rod (21). The sleeve (22) is secured on the first electrode rod (21) by bolts (24). The sleeve (22) has a second electrode rod (23) centrally downward extending therefrom and the second electrode rod (23) is electrically connected to the first electrode rod (21) when the sleeve (22) is secured on the first electrode rod (21). Consequently, the second electrode rod (23) of the present invention can be replaced to have various lengths and diameters such that the present invention can use different distances between the second electrode rod (23) and the electrode ring (26) to make different obvious electric shock effects. In addition, as shown in FIG. 3, the second electrode rod (23) obviously extends over the electrode ring (26) that can prevent the mist, caused by electric shock, from being collected on the bottom of the hand-held seat (20) and causing a short circuit. The first electrode rod (21), the second electrode rod (23) and the electrode ring (26) of the present invention are made of metal that has a good electric conductivity, such as platinum. The platinum is hard to be oxidized and does not discharge poison ions.

The present invention further comprises a culture tray (30) having multiple skirts (31) upwardly extending therefrom. Each skirt (31) has a conducting ring (28) received therein. The conducting ring (28) has a diameter corresponding to that of the electrode ring (26). The conducting ring (28) is previously disposed in each of the skirts (31) and the plant tissue solution is received within the connecting ring (28), then the present invention can execute an electric shock to the plant tissue solution in the conducting ring (28). By such a manner, the plant tissue will not be hurt and polluted during operating such that the operating time is shortened.

The replaceable annular electrode in accordance with the present invention can select a second electrode rod (23) that has a suitable length and diameter to the operation such that the present invention can use different distances between the second electrode rod (23) and the electrode ring (26) to make different obvious electric shock effects. The conducting ring (28) is received in the skirt (31) and directly conducted with the electrode ring (26) that can provide a direct electric shock to the plant tissue solution within the conducting ring (28). The present invention uses low-voltage impulse electrophoresis to make the DNA near the cell that is transgenic and high-voltage electroporation to make the DNA into the cell. In addition, the elongated second electrode rod (23) can prevent the mist, caused by electric shock, from being collected on the bottom of the hand-held seat (20) and causing a short circuit. Furthermore, the replaceable annular electrode can be mounted to a universal connector and adapted to a horizontal platform such that multiple hand-held seat (20) can execute electric shock at the same time and the operator does not need to touch the electrode. As a result, the electric shock efficiency and the safety are promoted. In addition, the culture tray (30) with the conducting ring (28) can be disposed on a condenser (not shown) for controlling the temperature of the electrode to prevent the plant tissue from being hurt due to a high temperature caused by electric shock. The annular electrode of the present invention has a use scope greater than that of the conventional tubular electrode. The annular electrode can transfer DNA, ribonucleic acid (RNA), protein, chemicals and the like to the plant tissue for promoting the effect of plant transgenic.

Although the invention has been explained in relation to its preferred embodiment, it is to be understood that many other possible modifications and variations can be made without departing from the spirit and scope of the invention as hereinafter claimed.

What is claimed is:

1. A replaceable annular electrode for transgenic, comprising a hand-held electrode seat having multiple metal rods downward extending therefrom, an electrode ring secured on a free end of each of the multiple metal rods, a first electrode rod centrally downward extending from the hand-held seat and a sleeve sleeved on the first electrode rod, the sleeve selectively secured on the first electrode rod, the sleeve having a second electrode rod centrally downward extending therefrom and the second electrode rod electrically connected to the first electrode rod when the sleeve is secured on the first electrode rod, the second electrode rod being replaced to have various lengths and diameters such that the replaceable annular electrode can use different distances between the second electrode rod and the electrode ring to make different obvious electric shock effects.

2. The replaceable annular electrode as claimed in claim 1, wherein the second electrode rod obviously extends over the electrode ring that can prevent the mist, caused by electric shock, from being collected on the bottom of the hand-held seat and causing a short circuit.

3. The replaceable annular electrode as claimed in claim 1, wherein the first electrode rod, the second electrode rod and the electrode ring are made of metal that has a good electric conductivity, such as platinum.

4. The replaceable annular electrode as claimed in claim 1 further comprising a culture tray having multiple skirts upwardly extending therefrom, each skirt having a conducting ring received therein for containing plant tissue solution, the conducting ring having a diameter corresponding to that of the electrode ring, the conducting ring directly conducted with the electrode ring that can provide a direct electric shock to the plant tissue solution within the conducting ring.

5. The replaceable annular electrode as claimed in claim 1, wherein the hand-held seat is adapted to be mounted to a universal connector such that multiple hand-held seats can execute electric shock at the same time and the operator does not need to touch the electrode.

6. The replaceable annular electrode as claimed in claim 4, wherein the culture tray with the conducting ring is adapted to be disposed on a condenser for controlling the temperature of the electrode to prevent the plant tissue from being hurt due to a high temperature caused by electric shock.
